(19) United States
(12) Plant Patent Application Publication
(10) Pub. No.: US 2019/0223341 P1
(43) Pub. Date: Jul. 18, 2019

(54) Raspberry plant - 'Dafne' cultivar

(50) ;

(71) Applicant: SANT'ORSOLA SOCIETA' COOPERTIVA AGRICOLA, Pergine Valsugana (IT)

(72) Inventors: Sara Giacomelli, Vigolo Vattaro (IT); Loris Osti, Tocegno (IT); Marco Giacomelli, Vigolo Vattaro (IT); Alessandra Viliotti, Vigolo Vattaro (IT)

(21) Appl. No.: 16/350,828

(22) Filed: Jan. 18, 2019

(30) Foreign Application Priority Data
Jan. 18, 2018 (QZ) 20180036

(51) Int. Cl. A01H 6/74

(52) U.S. Cl. PLT/204

(57) ABSTRACT

A new a primocane raspberry cultivar named ‘Dafne’ which produces glossy, strong red firm fruits is disclosed, with berries which are easy to pick due to low adherence to the plug. The new cultivar has the advantages of producing two crops per year, wherein the fruits equally ripen on the current season's cane in autumn and on the previous year's cane in summer.

Genus and species: Rubus idaeus L.

Variety denomination: ‘Dafne’.

### BACKGROUND OF THE NEW VARIETY

A new and distinct cultivar of red raspberry (Rubus idaeus L.) hereinafter referred to by the cultivar denomination ‘Dafne’ is disclosed. The new variety ‘Dafne’ was obtained from open pollination of the variety Amaranta (mother plant) and an unknown father plant pollen.

The crossing for the new cultivar occurred in year 2012 in Altopiano della Vigolana (Trento-Italy) and it was subjected to selection in 2013, and successively tested until 2017. The characteristics of the new cultivar have been found stable and have been transmitted without change through succeeding (more than 5 years) asexual propagations by root cutting.

Plant breeder's right were applied for with the European Union on Jan. 18, 2018. ‘Dafne’ has not been made publicly available.

### SUMMARY OF THE INVENTION

The most outstanding and distinguishing characteristics of the ‘Dafne’ cultivar are hereunder reported.
Primocane raspberry plant;
Good fruit colour and flavour;
Glossy strong red firm fruit;
Easy to pick up (low adherence to plug).

The new variety produces two crops per year and the fruits mainly ripen on current season's cane in autumn.

The new variety can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar ‘Dafne’ is illustrated by the accompanying photographs showing typical specimens of the new variety in colour as true as reasonably possible to make the same colour illustrations of this character by conventional photographic procedures.

FIG. 1 is a view showing the upright, tall and vigorous raspberry plants during the flowering.

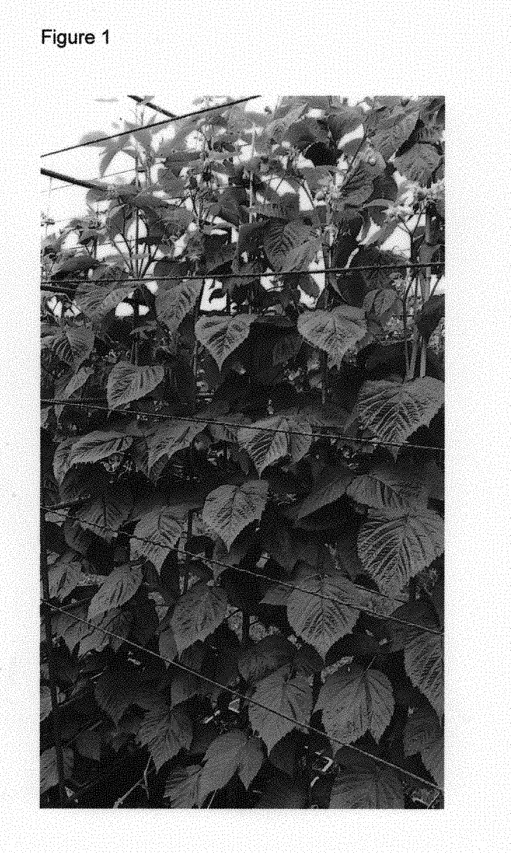

FIG. 2 is a view showing the leaf upper and lower surfaces.

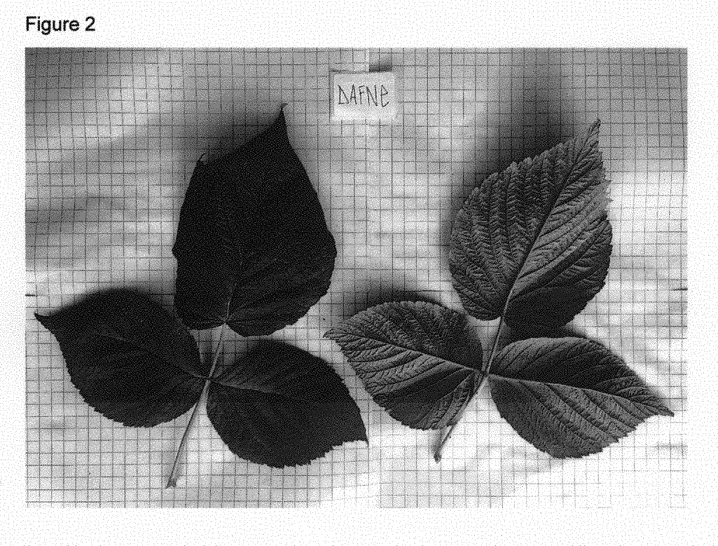

FIG. 3 is a view showing the plant primocane internode length, cane colour and thorns.

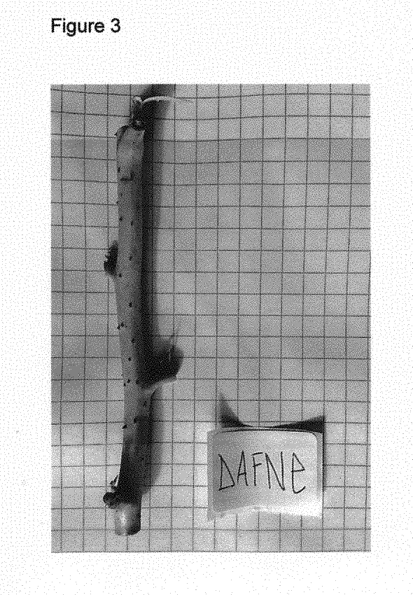

FIG. 4 is a view showing the ripe fruit size and shape.

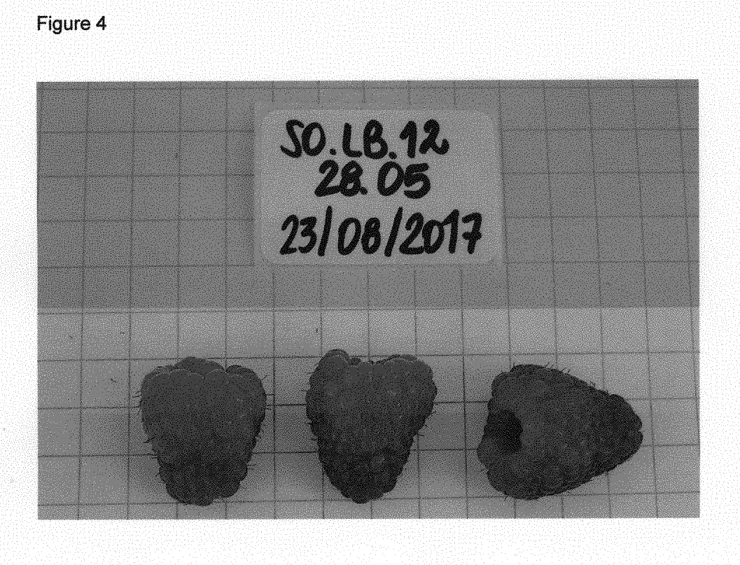

### DETAILED DESCRIPTION

The following detailed description set forth the distinctive characteristics of the new variety ‘Dafne’. The performance of new cultivar for retention of its distinctive characteristics has been evaluated through successive asexual propagations by root cutting in the Applicant's setting in Trentino (Italy) under natural conditions in high tunnels. The growing was in soilless using coir as substrate.

The plant assessment was performed for 2 years.
Classification:
   Family.—Rosaceae.
   Botanical.—Rubus idaeus L.
   Common name.—Raspberry.
Parentage: Amaranta (mother) x unknown pollen (father).
Plant:
   General habit.—Upright canes; strong vigor; very good roots system; adapted to medium winter chill conditions such as those of Northern Italy. Suitable for being harvested both on current year's cane in autumn and on previous year's cane in summer.
   Average height (average).—1.70-1.80 m.
   Spread (average).—0.80 m.
   Shape.—Conical.
   Vegetative bud length.—6.0-8.0 mm.
Primocane:
   Length (average).—1.70-1.80 m.
   Diameter (average).—1.0 cm.
   Texture.—Smooth.
   Internode length (average)at 1 m height.—5-6 cm.
   Number of fruiting laterals per cane (average).—9.
   Number of current season's shoots.—Medium.
   Young shoot colour.—*RHS:144A. Colour references made to R.H.S. Colour Chart of The Royal Horticultural Society of London (Edition V)
   Cane colour.—*RHS: N144C.
   Spines.—Present.
Floricane:
   Dormant cane colour.—*RHS: 72A.
   Fruiting lateral attitude in summer.—Semi erect.
Spines:
   Shape.—‘Shark fin’.
   Size (average).—1.50-1.70 mm.
   Density (average)at 1.2 m height.—2.3-2.5 cm².
   Colour.—*RHS N77A.
Foliage:
Leaves:
   Shape.—Compound; odd-pinnate and subdivided in 3 cordate and serrate.
   Profile in cross section.—Slightly convex.
   Rugosity.—Medium.
   Complete leaft.—Length: 34.2 cm. Width: 31.5 cm.
   Upper surface colour.—*RHA 141A.
   Lower surface colour.—*RHS 147D.
   Lateral leaflets.—Speculate.
   Lateral leaflets average size.—Length: 15.4 cm. Width:11.2 cm.
   Lateral leaflets apex shape.—Pointed.
   Lateral leaflets base shape.—Ovate.
   Lateral leaflets margin type.—Serrate.
   Terminal leaflets average size.—Length:16.9 cm. width:14.5 cm.
   Terminal leaflets apex shape.—Pointed.
   Terminal leaflets base shape.—Cordate.
   Terminal leaflets margin type.—Serrate.
Petiole:
   Average length.—10.8 cm.
Flowers:
   Number per plant (average).—Primocane: 160. Floricane: 195.
   Length (average).—9.66 mm.
   Diameter (average).—34.79 mm.
   Diameter (receptacle).—11.29 mm.
   Shape.—Pentagonal.
   Petal colour.—*RHS: NN155C.
   Fragrance.—Absent.
   Blooming.—Primocane: July-August. Floricane: April-May.
   Pedicel.—Length (average): 2.6 cm.
Fruit:
   Predominant shape.—Broad conical.
   Weight.—6.3 g.
   Size.—Length: 2.3 cm. width: 2.6 cm.
   Colour of ripe fruit.—*RHS: 50A.
   Colour of immature fruit.—*RHS: 52D.
   Firmness.—Firm (0.05 Kg).
   Drupes per fruit (average).—140.
   Drupe size.—Length: 4.07 mm. width: 3.5 mm. depth: 6.2 mm.
   Glossiness.—High.
   Adherence to plug.—Low.
   Titratable acidity.—10.24 meq/hg.
   Soluble solids.—9 °Brix.
   Harvest season.—Primocane: August to October. Floricane: half June-end July.
   Autumn yield.—4 kg/m (4 plants/m).
   Summer yield.—4.9 kg/m (4 plants/m).
   Storage capacity.—(Shelf life): good shelf-life.
Reproductive organs:
   Pistil number per flower (average).—151.
   Pistil size (average).—5.70 mm.
   Stamen number per flower (average).—150.
   Stamen size (average).—5.23 mm.
Seeds:
   Size (average).—Length: 2.83 mm. width: 1.71 mm.
   Shape.—Kidney-shaped.
Comparison with parental and commercial cultivar:

TABLE 1
Comparison table between variety and parents/other

| Characteristic | EVITA | LAGORAI PLUS | DAFNE |
|---|---|---|---|
| fruit color | Orange red | Strong red | strong red |
| Fruit: glossiness | low | medium | high |
| Fruit shape | elongated conical, | conical | broad conical, |
| | slight irregular | very regular | very regular |
| Fruit picking | Slight difficult | Very easy | very easy |
| Autumn yield | high | low | medium |
| Summer yield | | high | medium |
| Dormant cane colour | purple-brown | moderate brown | greyish-purple |

What is claimed:

1. A new and distinct cultivar of raspberry plant as substantially herein shown and described.
